(19) United States
(12) Patent Application Publication
(10) Pub. No.: US 2007/0167494 A1
(43) Pub. Date: Jul. 19, 2007

(54) Process for preparing a polymorph or rosiglitazone maleate

(75) Inventors: Andrew Simon Craig, Kent (GB); Robert Gordon Giles, Kent (GB); Jonathan Robert Saville Smithers, Kent (GB); Tim Chien Ting Ho, Kent (GB); Michael John Sasse, Kent (GB)

Correspondence Address: SMITHKLINE BEECHAM CORPORATION;CORPORATE INTELLECTUAL PROPERTY-US, UW2220, P. O. BOX 1539, KING OF PRUSSIA, PA, 19406-0939, US

(73) Assignee: Glaxo Group Limited

(21) Appl. No.: 10/551,021

(22) PCT Filed: Mar. 25, 2004
(86) PCT No.: PCT/GB04/01306

§ 371 Date: Feb. 26, 2007

(30) Foreign Application Priority Data
Mar. 28, 2003 (GB) 0307259.2

(51) Int. Cl. A61K 31/4439; C07D 417/02

(52) U.S. Cl. 514/342; 546/269.7

(57) ABSTRACT

A crystallization process for preparing a polymorph of rosiglitazone maleate (Compound 1), and a process for preparing Compound 1 with a polymorphic purity that is suitable for use as a seed material in a crystallization process for preparing Compound 1.

The present invention is concerned with the maleate salt of the antidiabetic 5-[4-[2-(N-methyl-N-(2-pyridyl)amino)ethoxy]benzyl]thiazolidine-2,4-dione, which has the approved name rosiglitazone and more particularly with its production and isolation.

Rosiglitazone which is described and claimed in EPA 0306228 shows good blood glucose lowering activity and is useful for the treatment and or prophylaxis of hyperglycemia and of particular use in the treatment of Type II diabetes, hyperlipidaemia, hypertension, cardiovascular disease and certain eating disorders.

An improved process for the preparation of rosiglitazone is described and claimed in EPA 121 9620A1.

EP0658161 B1 describes the preparation and isolation of a maleate salt of rosiglitazone, which is hereinafter referred to as Compound 1. More particularly EP0558161 B1 teaches that the maleate salt of rosiglitazone (Compound 1) may be prepared by dissolving rosiglitazone and maleic acid in hot ethanol, filtering the hot solution, allowing it to cool, and then filtering off the required salt which had then crystallised from the solution.

Subsequently three further polymorphs of rosiglitazone maleate were discovered and these are described in WO 00/64892, WO 00/64896 and WO 00/64893. These applications teach that Compound 1 may be prepared by dissolving each of the three polymorphs in hot denatured ethanol and then seeding with Compound 1. Thus WO 00/64893 teaches that Compound 1 may be prepared by dissolving the new polymorph described therein (and herein after referred to as the Form 4 polymorph) in hot denatured ethanol, filtering the hot solution into a preheated vessel (56°), heating the filtrate to 60° C., cooling with stirring, at 55° C. seeding with the Compound 1 and then the cooling process continued. WO00/64896 further teaches that Compound 1 may be prepared by dissolving the new Polymorph described therein in hot acetone, cooling to 50° C., seeding with Compound 1 and then the cooling process continued. For use in therapy the required pharmaceutical formulations of rosiglitazone are conveniently prepared using Compound 1 and therefore it is necessary that the process used for its manufacture is robust and consistently provides the desired product at a quality suitable for that use.

Prior to the preparation and isolation of the three additional polymorphs of rosiglitazone maleate the process described in EP0658161 B1 consistently met the requirements for producing, on a manufacturing scale, the required Compound 1 of a quality suitable for pharmaceutical use.

Subsequent to the preparation and isolation of the three additional polymorphs it was found that the described process no longer provided a reliable method for the preparation of Compound 1 and it was necessary to develop a more robust process for preparing the required Compound 1 on a commercial scale. (More specifically the method described was sometimes found to generate the Form 4 polymorph).

We have now found that the required Compound 1 of a quality suitable for pharmaceutical use can consistently be prepared by crystallisation of rosiglitazone maleate, without the need for seeding, in a solvent with a suitable dielectric constant.

The present invention thus provides a process for preparing the rosiglitazone maleate polymorph (Compound 1) substantially free of any other polymorphic forms which comprises crystallising rosiglitazone maleate in a solvent or mixture of solvents with a dielectric constant such that it provides Compound 1 substantially free of any other polymorphic forms.

The term substantially free as used herein refers to Compound 1 which preferably contains less than 10% of other polymorphs and more particularly approximately 5% or less of other polymorphs of rosiglitazone maleate. The amount of other polymorphs in Compound 1 can be determined using standard solid state analytical procedures such as X-ray powder diffractometry and infrared spectroscopy including infrared spectroscopy with second derivative processing. One embodiment of the present invention provides a process for preparing Compound 1 substantially free of other polymorphs, which comprises crystallising rosiglitazone maleate in a solvent with a dielectric constant of less than 21 or a mixture of solvents wherein at least one solvent has a dielectric constant of less than 21.

Suitable solvents with a dielectric constant of less than 21 for use in the crystallisation process include anisole, isopropyl acetate, ethyl acetate, dichloroethane, methyl isobutyl ketone, n-butanol, propan-2-ol, toluene, dimethyl carbonate, methyl ethyl ketone, acetone, or tetrahydrofuran or mixtures thereof. Further suitable solvents include mixtures of the abovementioned solvents (with dielectric constant<21) with other solvents, especially solvents with good solubility characteristics, for example ethanol, or denatured ethanol (Industrial Methylated Spirit [IMS]). For example suitable mixtures are ethyl acetate and IMS, or toluene and IMS, or dimethyl carbonate and IMS.

A particularly useful solvent for use in this process is tetrahydrofuran. The required solution of rosiglitazone maleate for use in the process may be obtained by heating rosiglitazone maleate in the chosen solvent, conveniently at a temperature of less than 70° C. Alternatively the required solution of rosiglitazone maleate may be obtained by combining rosiglitazone and maleic acid in the chosen solvent; conveniently at a temperature of less than 70° C.

When the required solution of rosiglitazone maleate for use in the process is obtained by heating rosiglitazone maleate in the chosen solvent preferably the hot solution is passed through a pre-heated filter prior to cooling the filtrate and then isolating the required Compound 1.

The Compound 1 prepared according to the process of the invention being substantially free of any other polymorphs of rosiglitazone maleate is therefore suitable for pharmaceutical use.

In a further aspect the invention provides a process for preparing the rosiglitazone maleate polymorph (Compound 1) essentially free of any other polymorphic forms, which comprises crystallising rosiglitazone maleate in a solvent or mixture of solvents with a dielectric constant such that it provides Compound 1 essentially free of any other polymorphic forms.

The term essentially free as used herein means that the Compound 1 does not contain any detectable levels of the other known polymorph forms of rosigiltazone maleate (i.e. less than 2%) when analysed by conventional techniques known for solid state analysis, conveniently X-ray diffraction techniques and or infrared spectroscopy including infrared spectroscopy with second derivative processing. More preferably the term ‘essentially free’ means that when the product of the process is used as seed material in a rosiglitazone maleate crystallisation (which without seeding would not furnish polymorphically pure Compound 1) the resultant Compound 1 also does not contain any detectable levels of any other polymorph when analysed by conventional solid state analytical procedures. Suitable solid state analysis procedures and techniques include infrared spectroscopy, X-ray diffraction techniques, Raman spectroscopy and Solid State Nuclear Magnetic Resonance. In particular, X-ray powder diffractometry and infrared spectroscopy including infrared spectroscopy with second derivative processing are suitable techniques.

One embodiment of this further aspect of the invention provides a process for preparing the Compound 1 essentially free of any other polymorphic forms of rosiglitazone maleate which comprises crystallising rosiglitazone maleate from a solvent or mixture of solvents wherein the solvent or at least one of the solvents has a dielectric constant of less than 14. Conveniently the solvent used in the crystallisation process have a dielectric constant of greater than 2.0 and less than 14.

Suitable solvents for use in the crystallisation process include anisole, isopropyl acetate, ethyl acetate, dichloroethane, methyl isobutyl ketone, dimethyl carbonate or tetrahydrofuran or mixtures thereof or mixtures with a solvent with a dielectric constant greater than 14 such as IMS. An example of such a suitable mixture is ethyl acetate and IMS.

A particularly useful solvent for use in this process is tetrahydrofuran.

The required solution of rosiglitazone maleate for use in the process may be obtained by heating rosiglitazone maleate in the chosen solvent, conveniently at a temperature of less than 70°. Alternatively the required solution of rosiglitazone maleate may be obtained by combining rosiglitazone and maleic acid in the chosen solvent, conveniently at a temperature of less than 70° C.

When the required solution of rosiglitazone maleate for use in the process is obtained by heating rosiglitazone maleate in the chosen solvent preferably the hot solution is passed through a pre-heated filter prior to cooling the filtrate and then isolating the required Compound 1. When the required solution of rosiglitazone maleate for use in the process is obtained by heating rosiglitazone free base and maleic acid in the chosen solvent the resultant hot solution is conveniently passed through a pre-heated filter prior to cooling the filtrate and then isolating the required compound 1. Conveniently the vessel collecting the filtrate is free of any contamination by any other polymorph and this may be achieved by washing procedures.

We have found that when Compound 1 essentially free of other polymorphs is used as seed material in the process for crystallisation of rosiglitazone maleate from a solution in a solvent with dielectric constant>21 such as ethanol e.g. denatured ethanol, the product of this process is Compound 1 of a quality suitable for pharmaceutical use.

The term ‘of a quality suitable for pharmaceutical use’ as used herein preferably refers to Compound 1 which is substantially free of other polymorphs and more preferably is essentially of other polymorphs.

Further this process is not only robust but provides a particularly advantageous means for preparing Compound 1 of the required quality on a commercial scale.

Thus in a further aspect the invention further provides a process for preparing Compound 1 which comprises seeding a solution of rosiglitazone maleate in a suitable solvent with a dielectric constant>21 with Compound 1 essentially free of other polymorphic forms prepared according to the invention.

Suitable solvents for use in this process include ethanol or denatured ethanol. In a preferred embodiment of this invention the process for preparing Compound 1 comprises seeding a solution of rosiglitazone maleate in denatured ethanol (IMS) with Compound 1 seed material prepared according to the invention. Conveniently this process is carried out by heating the solution of rosiglitazone maleate in denatured ethanol to a temperature of less than 70° C. e.g. 68-69°, adjusting the temperature of the filtrate to approximately 60° C. cooling with stirring, then adding the seed material when the solution temperature is approximately 50° and then continuing the cooling to a temperature of less than 25° C. and isolating the Compound 1 by filtration. A preferred aspect of this process is when the seed material is that prepared by crystallisation from tetrahydrofuran.

The invention further provides a process for the preparation of Compound 1, essentially free from any other polymorph of rosiglitazone maleate which comprises crystallising rosiglitazone maleate from a solvent selected from anisole, isopropyl acetate, ethyl acetate, dichloroethane, dimethyl carbonate, methyl isobutyl ketone or tetrahydrofuran or mixtures thereof or a mixture of ethyl acetate and denatured ethanol (IMS).

The required solution of rosiglitazone maleate for use in the process may be obtained by heating rosiglitazone maleate in the chosen solvent, conveniently at a temperature of less than 70° C.

The process according to the invention is preferably carried out by filtering the hot solution through a pre-heated filter, cooling the filtrate and then collecting the required Compound 1 by filtration. Conveniently the vessel collecting the filtrate is free of any contamination with any other polymorph of rosiglitazone maleate and this may be achieved by conventional cleaning procedures.

Alternatively the solution of rosiglitazone maleate may be prepared by mixing rosiglitazone free base with maleic acid in the chosen solvent with heating if appropriate and then subsequent cooling of the heated solution.

A particularly useful solvent for use in this process is tetrahydrofuran.

The characterising data for the polymorph of rosiglitazone maleate referred to herein as Compound 1 is given below:

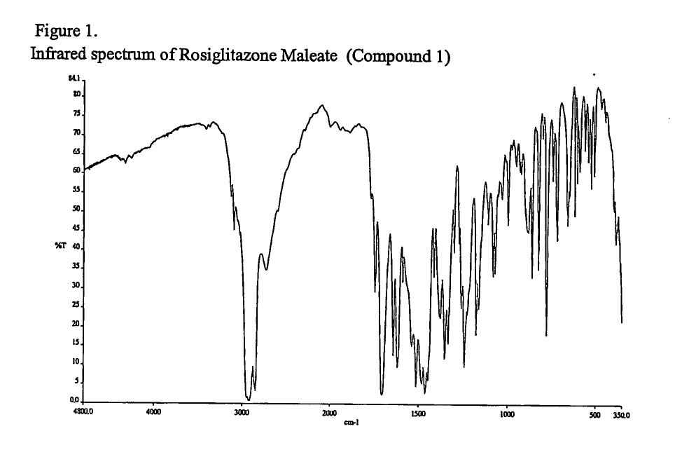

The infrared absorption spectrum of a mineral oil dispersion of the product was obtained using a Nicolet 710 FT-IR spectrometer at 2 cm⁻resolution (FIG. 1). Data were digitised at 1 cm⁻¹ intervals. Bands were observed at: 4327, 3420, 3131, 3099, 2950, 2924, 2853, 2732, 1889, 1744, 1705, 1640, 1617, 1586, 1538, 1513, 1482, 1463, 1449, 1414, 1384, 1377, 1353, 1335, 1303, 1274, 1262, 1245, 1227, 1179, 1164, 1109, 1083, 1070, 1030, 997, 952, 933, 924, 902, 882, 861, 823, 801, 778, 742, 723, 718, 657, 647, 617, 605, 590, 560, 541, 525, 508, 467, 445, 396, 384, 373, 367, 360, 357 cm⁻¹.

XRPD for Rosiglitazone Maleate (Compound 1)

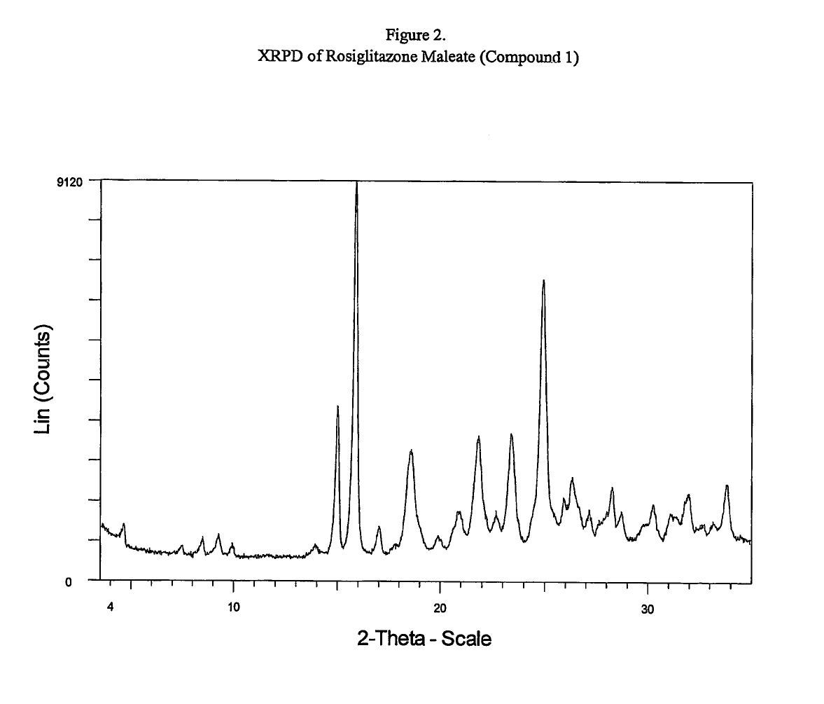

The XRPD pattern of the product (FIG. 2) was recorded using the following acquisition conditions: Tube anode: Cu, Generator tension: 40 kV, Generator current: 30 mA, Start angle: 3.5 °2θ, End angle: 35.0 °2θ, Step size: 0.02 °2θ, Time per step: 4.55 seconds. Characteristic XRPD angles and relative intensities are recorded in Table 1.

TABLE 1

| Angle 2-Theta° | Rel. Intensity % |
|---|---|
| 4.6 | 14.0 |
| 7.4 | 8.5 |
| 8.4 | 10.7 |
| 9.2 | 10.8 |
| 9.9 | 9.1 |
| 13.9 | 9.0 |
| 15.0 | 43.7 |
| 15.9 | 100.0 |
| 17.0 | 13.5 |
| 17.8 | 9.2 |
| 18.6 | 32.8 |
| 19.9 | 11.2 |
| 20.6 | 13.2 |
| 20.9 | 17.3 |
| 21.8 | 36.3 |
| 22.7 | 17.5 |
| 23.4 | 36.9 |
| 24.9 | 75.5 |
| 26.0 | 20.7 |
| 26.3 | 25.9 |
| 26.7 | 18.6 |
| 27.2 | 17.9 |
| 27.7 | 14.5 |
| 28.3 | 23.5 |
| 28.7 | 17.3 |
| 29.8 | 14.3 |
| 30.3 | 19.2 |
| 31.1 | 16.9 |
| 31.4 | 16.3 |
| 32.0 | 22.0 |
| 32.7 | 14.1 |
| 33.2 | 14.4 |
| 33.9 | 24.3 |

The characterising data for the other known polymorphs of rosiglitazone maleate are described in WO 00/64892, WO 00/64896 and WO 00/64893.

The following examples illustrate the invention but does not limit it in any way.

The dielectric constant values (determined at 20° C.) of the solvents used in the example are as follows:—
Toluene (2.4), anisole (4.3), diethyl ether (4.3), ethyl acetate (6.0), tetrahydrofuran (7.6), dichloroethane (10.4), methyl isobutyl ketone (13.1), n-butanol (17.5), propan-2-ol (18.3) methyl ethyl ketone (18.5), acetone (20.6) and ethanol (22.4), [Ian M Smallwood (1996) Handbook of Organic Solvent Properties, Arnold, London] Dimethylcarbonate (3.2) [H. D. Goodfellow and W. F. Graydon, Chemical Engineering Science, 1968, Vol 23, pp. 1267-12810 Pergamon Press, GB], Isopropyl acetate (4.7) [C Mialkowski, A Chagnes, B Carré, D Lemordant and P Willmann, J Chem. Thermodynamics, 2002, 34, 1847-1856].

Unless otherwise stated, the polymorphic purity of the ‘Compound 1’ obtained in the examples was determined using infrared, the absorption spectrum being obtained from a mineral oil dispersion of the compound using a Nicolet 710 FT-IR spectrometer at 2 cm⁻¹ resolution or of the solid product using a Perkin-Elmer Spectrum One FT-IR spectrometer fitted with a universal ATR accessory.

Unless otherwise specified in the examples the rosiglitazone maleate used as input material was the polymorph herein before identified as Compound 1.

### SECTION A

### Preparations of Compound 1 (Rosiglitazone Maleate) Essentially Free of Other Polymorphs.

### Example 1

Rosiglitazone maleate (1.0 g) was added to anisole (200 ml) and the mixture was heated to 70° C., then filtered to remove undissolved material. The filtrate was reheated to 65° C. and allowed to cool. The mixture was stirred for 2 hours at 20-25° C. then filtered, the filter cake washed with diethyl ether (10 ml), and the solid dried in a vacuum oven to give Compound 1 (0.25 g).

### Example 2

Rosiglitazone maleate (2.0 g) was added to isopropyl acetate (400 ml) and the mixture was heated to 75° C., then filtered to remove undissolved material. The filtrate was reheated to 65° C. and allowed to cool. The mixture was stirred for 2 hours at 20-25° C. then filtered. The filter cake washed with isopropyl acetate (10 ml), and the solid dried in a vacuum oven to give Compound 1 (1.32 g).

### Example 3

Rosiglitazone maleate (2.0 g) was added to ethyl acetate (200 ml) and the mixture was heated to reflux, and the resulting solution was filtered. The filtrate was reheated to reflux and allowed to cool. The resulting suspension was stirred for 2 hours at 20-25° C. then filtered. The filter cake washed with ethyl acetate (10 ml), and dried in a vacuum oven to give Compound 1 (1.58 g).

### Example 4A

Rosiglitazone maleate (5.0 g) was added to tetrahydrofuran (35 ml) and the mixture was heated to reflux, then filtered. The filtrate was reheated to reflux and allowed to cool. The mixture was stirred for 1.5 hours at 20-25° C. then filtered. The filter cake washed with tetrahydrofuran (8 ml), and the solid dried in a vacuum oven to give Compound 1 (3.56 g)

### Example 4B

Rosiglitazone maleate (5.0 g) was added to tetrahydrofuran (100 ml) and the mixture was heated to reflux to give a solution, and then filtered. The filtrate was transferred to a pre-heated vessel via an inline filter under nitrogen pressure. Tetrahydrofuran was distilled off until a residual volume of 35-40 ml remained. The solution was cooled to 20° C. resulting in crystallisation. The mixture was stirred for 2 hours at 20° C. and the product was filtered, washed with tetrahydrofuran (5 ml) and dried at 50° C. to give Compound 1 (3.55 g)

### Example 5

Rosiglitazone maleate (2.0 g) was added to dichloroethane (85 ml) and the mixture was heated to reflux, then filtered. The filtrate was reheated to 70° C. and allowed to cool. An oil was originally produced which crystallised upon further cooling. The mixture was stirred for 2 hours at 20-25° C. then filtered. The filter cake dried in a vacuum oven to give Compound 1 (1.67 g).

### Example 6

Rosiglitazone maleate (2.0 g) was added to methylisobutyl ketone (240 ml) and the mixture was heated to 70° C., then filtered. The filtrate was reheated to 65° C. and allowed to cool. Crystallisation commenced after 0.5 hours at 20-25° C.—the mixture was stirred for a further 1.5 hours at 20-25° C. then filtered. The filter cake was washed with methylisobutyl ketone (15 ml), and dried in a vacuum oven to give Compound 1 (1.33 g).

### Example 7

A mixture of rosiglitazone free base (6.0 g) and tetrahydrofuran (30 ml) was heated to 35° C., and maleic acid (2.10 g) was added. The resulting solution was heated to 60° C., held at this temperature for 20 min, then filtered. The filtrate was reheated to 60° C. and allowed to cool. The mixture was stirred for 2 hours at 20-25° C. then filtered. The filter cake washed with tetrahydrofuran (10 ml) and dried in a vacuum oven to give compound 1 (5.22 g).

### Example 8

Maleic acid (3.3 g) was added to a stirred suspension of rosiglitazone (10.0 g) in tetrahydrofuran (100 ml). The reaction mixture was stirred for 45 minutes at 21° C. The clear solution was filtered, reduced to 50 ml, then stirred for 17 hours at 21° C. The white solid was collected by filtration, washed with tetrahydrofuran (20 ml) then dried on the filter for 15 minutes to give the product as a white solid (11.65 g)

### Example 9

Maleic acid (0.33 g) was added to a stirred suspension of rosiglitazone (1.0 g) in diethyl ether (200 ml) at 21° C. The reaction mixture was stirred at reflux for 30 minutes, then cooled to 21° C. (A clear solution was not observed) The reaction mixture was stirred for 24 hours at 21° C., the white solid was collected by filtration, washed with diethyl ether (20 ml) then dried on the filter for 15 minutes to give the product as a white solid (1.1 g)

### Example 10

Maleic acid (0.33 g) was added to a stirred suspension of rosiglitazone (1.0 g) in a pre-mixed solvent mixture of IMS: ethyl acetate (3 ml: 7 ml) at 21° C. under argon. The reaction mixture was heated at an oil bath temperature of 55° C. for 30 minutes, then cooled to 21° C. and stirred for 17 hours at 21° C. The white solid was collected by filtration, washed with IMS (20 ml) then dried on the filter for 15 minutes to give the product as a white solid (0.97 g)

### Example 11

Maleic acid (0.33 g) was added to a stirred suspension of rosiglitazone (1.0 g) in dichloroethane (50 ml) at 21° C. The reaction mixture was heated at an oil bath temperature of 76° C. for 30 minutes. The clear solution was cooled to 21° C. and stirred for 150 minutes. The white solid was collected by filtration, washed with dichloroethane (10 ml) then dried on the filter for 20 minutes to give the product as a white solid (1.14 g)

### Example 12

Rosiglitazone maleate (Form 4 polymorph 1.0 g) in tetrahydrofuran (15 ml) was heated for 24 minutes at reflux (oil bath temperature of 79° C.). The hot clear solution was cooled to 21° C. with stirring. Stirring was continued for a further 17.5 hours at 21° C. The white solid was collected by filtration, washed with tetrahydrofuran (5 ml) then dried under vacuum over phosphorus pentoxide for 2 hours at 21° C. to give the product as a white solid (0.44 g).

### Example 13

Rosiglitazone maleate (Form 4 polymorph 1.0 g) and 1,2-dichloroethane (50 ml) was heated at an oil bath temperature of 79° C. with stirring. Additional volumes of 1,2-dichloroethane were added after 20 minutes (25 ml) and 30 minutes (25 ml) respectively. The resulting suspension was heated at an oil bath temperature of 79° C. with stirring for 30 minutes, then filtered. The clear filtrate was stirred for 16 hours at 21° C. The white solid was collected by filtration, washed with 1,2-dichloroethane (5 ml) then dried on the filter for 20 minutes to give the product as a white solid (0.55 g)

### Example 14

Rosiglitazone maleate (Form 4 polymorph (1.0 g) and ethyl acetate (100 ml) was heated at an oil bath temperature of 79° C. with stirring. Additional volumes of ethyl acetate were added after 20 minutes (50 ml) and 30 minutes (50 ml) respectively. The resulting suspension was heated at an oil bath temperature of 79° C. with stirring for 25 minutes, then filtered. The dear filtrate was stirred for 16 hours at 21° C. The white solid was collected by filtration, washed with ethyl acetate (5 ml), dried on the filter for 20 minutes to give the product as a white solid (0.52 g).

Solid state infrared spectral and or XRPD analysis of the products of Examples 1 to 14 and 22 to 24 did not find any detectable levels other polymorphs of rosiglitazone maleate.

### SECTION B

### Preparations of Compound 1 (Rosiglitazone Maleate) Substantially Free of Other Polymorphs

### Example 15

Rosiglitazone (3.33 g) in n-butanol (100 ml) was heated to 70° C. for 15 minutes, then filtered. The solution was reheated to 70° C., then cooled to 20-25° C. and stirred for 2 hours at 20-25° C. The white solid was collected by filtration, washed with IMS (8 ml) then dried at 50° C. under vacuum for 24 hours to give the product as a white solid (2.74 g).

Polymorphic purity approximately 95%.

### Example 16

Rosiglitazone (4.0 g) in methyl ethyl ketone (120 ml) was heated to 65-70° C. for 20 mins then filtered. The filtrate was reheated to 65° C., cooled to 20-25° C. and stirred for 2.5 hours at 20-25° C. The solid was collected by filtration, washed with methyl ethyl ketone (15 ml) then dried under vacuum at 50° C. for 18 hours to give the product as a white solid (2.42 g)

Polymorphic purity approximately 95%

### Example 17

Maleic acid (0.33 g) was added to a suspension of rosiglitazone (1.0 g) in propan-2-ol (20 ml) at 21° C. The mixture was stirred for 25 minutes at an oil bath temperature of 60° C., then cooled to 21° C. and stirred for 2 hours at 21° C. The white solid was collected by filtration, washed with IPA (10 ml) then dried on the filter for 10 minutes to give the product as a white solid (1.24 g).

Polymorphic purity>95%

### Example 18

Maleic acid (0.35 g) was added to a stirred suspension of rosiglitazone (1.0 g) in a mixture of IMS (10 ml) and toluene (25 ml) at 21° C. under argon. The reaction mixture was heated at an oil bath temperature of 55° C. for 30 minutes, then cooled to 21° C. and stirred for 17 hours 21° C. The white solid was collected by filtration, washed with toluene (10 ml) then dried on the filter for 10 minutes to give the product as a white solid (0.91 g)

Polymorphic purity>95%

### Example 19

Maleic acid (0.33 g) was added to a stirring suspension of rosiglitazone (1.0 g) in a pre-mixed solvent of IMS:dimethylcarbonate (5 ml:5 ml) at 21° C. under argon. The reaction mixture was heated at an oil bath temperature of 55° C. for 20 minutes, then cooled to 21° C. and stirred for 3 hours at 21° C. The white solid was collected by filtration, washed with IMS (20 ml) then dried on the filter for 20 minutes to give the product as a white solid (0.69 g)

Polymorphic purity approximately 95%

### Example 20

Maleic acid (0.32 g) was added to a stirred suspension of rosiglitazone (1.0 g) in acetone (20 ml) at 21° C. The reaction mixture was stirred at reflux for 30 minutes, then cooled to 21° C. with stirring. Crystallisation was observed after 30 minutes. The reaction was stirred for a further 16 hours at 21° C. The white solid was collected by filtration, washed with acetone (10 ml) then dried on the filter for 30 minutes to give the product as a white solid (0.9 g).

Polymorphic purity>95%

### Example 21

Rosiglitazone maleate (Form 4 polymorph 1.0 g) in acetone (30 ml) was heated for 20 minutes at reflux. The hot clear solution was filtered then cooled to 21° C. with stirring. Crystallisation was observed after 1 hour 55 minutes, stirring was continued for a further 19 hours. The white solid was collected by filtration, then dried under vacuum, over phosphorus pentoxide for 2 hours at 21° C. to give the white product as a white solid (0.51 g).

Polymorphic purity>95%

### SECTION C

### Process to Prepare Compound 1 Essentially Free of Other Polymorphs Using Suitable Seed Material

### Example 22

Maleic acid (0.33 g) was added to a stirred suspension of rosiglitazone (1.0 g) in IMS (30 ml). The reaction mixture was stirred at an oil bath temperature of 60° C. for 22 minutes. The hot solution was filtered, then seeded with the product of Example 8 (40 mg) and stirred for 2 hours at 21° C. The white solid was collected by filtration, washed with IMS (10 ml) and dried on the filter for 15 minutes to give the required product as a white solid (0.79 g).

### Example 23

A mixture of rosiglitazone (7.5 g) and maleic acid (2.55 g) was heated to 70° C. in industrial methylated spirit (75 mL) under nitrogen. After 30 minutes the clear solution was transferred to a pre-heated vessel via an in-line filter under nitrogen pressure. The solution was reheated to 70° C. with stirring and then cooled to 55° C. before being seeded with Compound 1 (0.3 g, prepared as in Example 4B). The mixture was cooled to 20° and stirred for 1 hour. The product was filtered, washed with industrial methylated spirit and dried to give Compound 1 (8.66 g, 85%)

### Example 24

Maleic acid (0.33 g) was added to a stirred suspension of rosiglitazone (1.0 g) in IMS (30 ml). The reaction mixture was stirred at an oil bath temperature of 60° C. for 30 minutes. The hot solution was filtered, then seeded with the product of Example 10 (40 mg) and stirred for 2 hours at 21° C. The white solid was collected by filtration, washed with IMS (10 ml) and dried on the filter for 15 minutes to give the product as a white solid (0.84 g).

1. A process for preparing the rosiglitazone maleate polymorph (Compound 1) substantially free of any other polymorphic forms, which comprises crystallizing rosiglitazone maleate in a solvent or mixture of solvents with a dielectric constant such that it provides Compound 1 substantially free of any other polymorphic forms.

2. A process for preparing the rosiglitazone maleate polymorph, Compound 1 substantially free of other polymorphs, which comprises crystallizing rosiglitazone maleate in a solvent with a dielectric constant of less than 21 or a mixture of solvents wherein at least one solvent has a dielectric constant of less than 21.

3. A process as claimed in claim 1 wherein the solvent is selected from anisole, isopropyl acetate, ethyl acetate, dichloroethane, methyl isobutyl ketone, n-butanol, propan-2-ol, toluene, dimethylcarbonate, or tetrahydrofuran or mixtures thereof.

4. A process as claimed in claim 1 wherein the crystallization solvent is a mixture selected from a ethyl acetate and IMS, toluene and IMS for dimethylcarbonate and IMS.

5. A process for preparing the rosiglitazone maleate polymorph (Compound 1) essentially free of any other polymorphic forms, which comprises crystallizing rosiglitazone maleate in a solvent or mixture of solvents with a dielectric constant such that it provides Compound 1 essentially free of any other polymorphic forms.

6. A process for preparing the Compound 1 essentially free of any other polymorphic forms of rosiglitazone maleate which comprises crystallizing rosiglitazone maleate from a solvent or mixture of solvents wherein the solvent or at least one of the solvents has a dielectric constant of less than 14.

7. A process as claimed in claim 5 for preparing the Compound 1 essentially free of any other polymorphic forms of rosiglitazone maleate which comprises crystallizing rosiglitazone maleate from a solvent or mixture of solvents with a dielectric constant of less than 14.

8. A process as claimed in claim 6 wherein the solvent has a dielectric constant of greater than 2.8 and less than 14.

9. A process as claimed in claim 5 wherein the solvent is tetrahydrofuran.

10. A process for preparing Compound 1 which comprises seeding a solution of rosiglitazone maleate in a suitable solvent with a dielectric constant>21, with Compound 1 essentially free of other polymorphic forms prepared according to the process as claimed in claim 5.

11. A process as claimed in claim 10 wherein the solvent is denatured ethanol.

12. (canceled)

13. A process for preparing the Compound 1 essentially free of any other polymorphic forms of rosiglitazone maleate which comprises crystallizing rosiglitazone maleate from a solvent or mixture of solvents selected from anisole, isopropyl acetate, ethyl acetate, dichloroethane, dimethyl carbonate, methyl isobutyl ketone, tetrahydrofuran or a mixture ethyl acetate and denatured ethanol (IMS).
